(19) United States
(12) Patent Application Publication
(10) Pub. No.: US 2019/0117787 A1
(43) Pub. Date: Apr. 25, 2019

(54) COMBINATIONS OF CD33 ANTIBODY DRUG CONJUGATES WITH HYPOMETHYLATING AGENTS

(71) Applicant: SEATTLE GENETICS, INC., Bothell, MA (US)

(72) Inventors: Dana Kennedy, Kirkland, WA (US); Megan O'Meara, Seattle, WA (US); Eric J. Feldman, Shoreline, WA (US)

(73) Assignee: SEATTLE GENETICS, INC., Bothell, WA (US)

(21) Appl. No.: 16/093,947

(22) PCT Filed: Apr. 12, 2017
(86) PCT No.: PCT/US2017/027246

§ 371 Date: Oct. 15, 2018

Related U.S. Application Data
(60) Provisional application No. 62/323,206, filed on Apr. 15, 2016.
(60) Provisional application No. 62/438,376, filed on Dec. 22, 2016.

(51) Int. Cl. A61K 47/68; A61K 31/706; C07K 16/28; A61K 31/5517; A61P 35/02

(52) U.S. Cl. CPC A61K 47/6803; A61K 31/706; C07K 16/2803; C07K 2317/24; A61K 31/5517; A61P 35/02; A61K 47/6849

(57) ABSTRACT

This invention relates to treatment of cancer using a CD33 antibody drug conjugate in combination with hypomethylating agents.

### CROSS-REFERENCES TO RELATED APPLICATIONS

This application claims the benefit of U.S. Provisional Application No. 62/323,206 filed Apr. 15, 2016 and U.S. Provisional Application No. 62/438,376 filed Dec. 22, 2016, each of which are incorporated by reference herein in its entirety.

### REFERENCE TO A SEQUENCE LISTING

This application includes an electronic sequence listing in a file named 0033-00314PC Sequence Listing ST25, created on Apr. 3, 2017 of 15 KB, which is herein incorporated by reference.

### FIELD OF THE INVENTION

This invention relates to treatment of cancer using a CD33 antibody drug conjugate in combination with hypomethylating agents.

### BACKGROUND OF THE INVENTION

CD33 is a 67 kDa plasma membrane protein that binds to sialic acid and is a member of the sialic acid-binding Ig-related lectin (SIGLEC) family of proteins. CD33 is known to be expressed on myeloid cells. CD33 expression has also been reported on a number of malignant cells. A clinical trial of a CD33 antibody drug conjugate, comprising an h2H12 antibody conjugated to a PBD molecule, has been initiated. Additional improvements in treatment of CD33 expressing cancers are being sought. The present invention solves these and other problems.

### BRIEF SUMMARY OF THE INVENTION

This disclosure provides a method of treating a CD33 expressing cancer by administering a CD33 antibody drug conjugate (ADC) and a hypomethylating agent. The CD33-ADC comprises a humanized 2H12 antibody and a PBD cytotoxic agent. The variable region sequences of the h2h12 antibody are SEQ ID NOs:1 and 2. Exemplary hypomethylating agents are 5-azacytidine and 5-aza-2-deoxycytidine. The CD33 expressing cancer is acute myeloid leukemia (AML).

The PBD cytotoxic agent has the formula

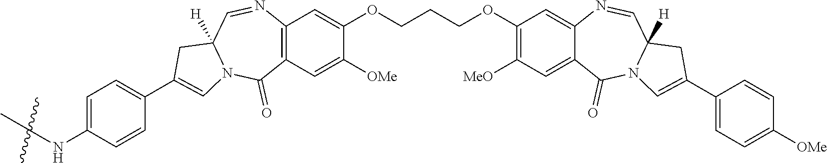 [SMILES: *Nc1ccc(C2=CN3C(=O)c4cc(OC)c(OCCCOc5cc6c(cc5OC)C(=O)N5C=C(c7ccc(OC)cc7)C[C@H]5C=N6)cc4N=C[C@@H]3C2)cc1]

A formula of the h2H12 antibody conjugated to the PBD molecule, including a linker has the formula

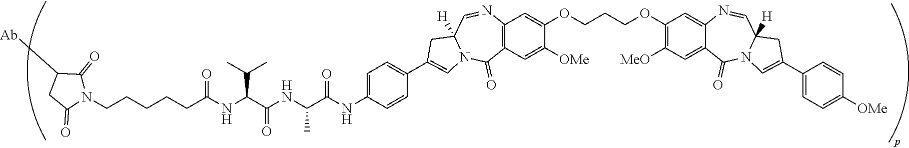

where h2H12 is denoted Ab.

The CD33-ADC is administered at a concentration of 10 μg/kg.

In some embodiments, the hypomethylating agent is 5-azacytidine. In one embodiment, the 5-azacytidine is administered at a concentration between 50-100 mg/m². In another embodiment, the 5-azacytidine is administered at a concentration of about 75 mg/m². In a further embodiment the 5-azacytidine is administered at a concentration of 75 mg/m².

In another embodiment, the CD33-ADC is administered at a concentration of 10 μg/kg in combination with 5-azacytidine, which is administered at a concentration of 75 mg/m².

In some embodiments, the hypomethylating agent is 5-aza-2-deoxycytidine. In one embodiment, 5-aza-2-deoxycytidine is administered at a concentration between 15-25 mg/m². In another embodiment, 5-aza-2-deoxycytidine is administered at a concentration of about 20 mg/m². In a further embodiment, 5-aza-2-deoxycytidine is administered at a concentration of 20 mg/m².

In another embodiment, the CD33-ADC is administered at a concentration of 10 μg/kg in combination with 5-aza-2-deoxycytidine, which is administered at a concentration of 20 mg/m².

In another embodiment, the CD33-ADC is administered at a concentration of 10 μg/kg in combination with 5-azacytidine, which is administered at a concentration of 75 mg/m² to a subject who is more than 75 years old and who has CD33-positive AML.

In another embodiment, the CD33-ADC is administered at a concentration of 10 μg/kg in combination with 5-aza-2-deoxycytidine, which is administered at a concentration of 20 mg/m² to a subject who is more than 75 years old and who has CD33-positive AML.

In another embodiment, the CD33-ADC is administered at a concentration between 20-40 μg/kg in combination with 5-aza-2-deoxycytidine, which is administered at a concentration of 20 mg/m² to a subject who is less than 75 years old and who has CD33-positive relapsed/refractory AML or to a subject who is less than 75 years old and who has CD33-positive secondary AML.

In another embodiment, the CD33-ADC is administered at a concentration of 20 μg/kg in combination with 5-aza-2-deoxycytidine, which is administered at a concentration of 20 mg/m² to a subject who is less than 75 years old and who has CD33-positive relapsed/refractory AML or to a subject who is less than 75 years old and who has CD33-positive secondary AML.

In another embodiment, the CD33-ADC is administered at a concentration of 30 μg/kg in combination with 5-aza-2-deoxycytidine, which is administered at a concentration of 20 mg/m² to a subject who is less than 75 years old and who has CD33-positive relapsed/refractory AML or to a subject who is less than 75 years old and who has CD33-positive secondary AML.

In another embodiment, the CD33-ADC is administered at a concentration of 40 μg/kg in combination with 5-aza-2-deoxycytidine, which is administered at a concentration of 20 mg/m² to a subject who is less than 75 years old and who has CD33-positive relapsed/refractory AML or to a subject who is less than 75 years old and who has CD33-positive secondary AML.

### Definitions

A “polypeptide” or “polypeptide chain” is a polymer of amino acid residues joined by peptide bonds, whether produced naturally or synthetically. Polypeptides of less than about 10 amino acid residues are commonly referred to as “peptides.”

A “protein” is a macromolecule comprising one or more polypeptide chains. A protein may also comprise non-peptidic components, such as carbohydrate groups. Carbohydrates and other non-peptidic substituents may be added to a protein by the cell in which the protein is produced, and will vary with the type of cell. Proteins are defined herein in terms of their amino acid backbone structures; substituents such as carbohydrate groups are generally not specified, but may be present nonetheless.

The terms “amino-terminal” and “carboxyl-terminal” are used herein to denote positions within polypeptides. Where the context allows, these terms are used with reference to a particular sequence or portion of a polypeptide to denote proximity or relative position. For example, a certain sequence positioned carboxyl-terminal to a reference sequence within a polypeptide is located proximal to the carboxyl terminus of the reference sequence, but is not necessarily at the carboxyl terminus of the complete polypeptide.

The term “antibody” is used herein to denote immunoglobulin proteins produced by the body in response to the presence of an antigen and that bind to the antigen, as well as antigen-binding fragments and engineered variants thereof. Hence, the term “antibody” includes, for example, intact monoclonal antibodies comprising full-length immunoglobulin heavy and light chains (e.g., antibodies produced using hybridoma technology) and antigen-binding antibody fragments, such as F(ab′)2 and Fab fragments. Genetically engineered intact antibodies and fragments, such as chimeric antibodies, humanized antibodies, single-chain Fv fragments, single-chain antibodies, diabodies, minibodies, linear antibodies, multivalent or multispecific (e.g., bispecific) hybrid antibodies, and the like are also included. Thus, the term “antibody” is used expansively to include any protein that comprises an antigen-binding site of an antibody and is capable of specifically binding to its antigen.

The term “genetically engineered antibodies” means antibodies wherein the amino acid sequence has been varied from that of a native antibody. Because of the relevance of recombinant DNA techniques in the generation of antibodies, one need not be confined to the sequences of amino acids found in natural antibodies; antibodies can be redesigned to obtain desired characteristics. The possible variations are many and range from the changing of just one or a few amino acids to the complete redesign of, for example, the variable or constant region. Changes in the constant region will, in general, be made in order to improve or alter characteristics such as, e.g., complement fixation, interaction with cells, and other effector functions. Typically, changes in the variable region will be made in order to improve the antigen-binding characteristics, improve variable region stability, or reduce the risk of immunogenicity.

An “antigen-binding site of an antibody” is that portion of an antibody that is sufficient to bind to its antigen. The minimum such region is typically a variable domain or a genetically engineered variant thereof. Single-domain binding sites can be generated from camelid antibodies (see Muyldermans and Lauwereys, J. Mol. Recog. 12:131-140, 1999; Nguyen et al., EMBO J. 19:921-930, 2000) or from VH domains of other species to produce single-domain antibodies (“dAbs”; see Ward et al., Nature 341:544-546, 1989; U.S. Pat. No. 6,248,516 to Winter et al.). In certain variations, an antigen-binding site is a polypeptide region having only 2 complementarity determining regions (CDRs) of a naturally or non-naturally (e.g., mutagenized) occurring heavy chain variable domain or light chain variable domain, or combination thereof (see, e.g., Pessi et al., Nature 362:367-369, 1993; Qiu et al., Nature Biotechnol. 25:921-929, 2007). More commonly, an antigen-binding site of an antibody comprises both a heavy chain variable (VH) domain and a light chain variable (VL) domain that bind to a common epitope. Within the context of the present invention, an antibody may include one or more components in addition to an antigen-binding site, such as, for example, a second antigen-binding site of an antibody (which may bind to the same or a different epitope or to the same or a different antigen), a peptide linker, an immunoglobulin constant region, an immunoglobulin hinge, an amphipathic helix (see Pack and Pluckthun, Biochem. 31:1579-1584, 1992), a non-peptide linker, an oligonucleotide (see Chaudri et al., FEBS Letters 450:23-26, 1999), a cytostatic or cytotoxic drug, and the like, and may be a monomeric or multimeric protein. Examples of molecules comprising an antigen-binding site of an antibody are known in the art and include, for example, Fv, single-chain Fv (scFv), Fab, Fab′, F(ab′)2, F(ab)c, diabodies, dAbs, minibodies, nanobodies, Fab-scFv fusions, bispecific (scFv)4-IgG, and bispecific (scFv)2-Fab. (See, e.g., Hu et al., Cancer Res. 56:3055-3061, 1996; Atwell et al., Molecular Immunology 33:1301-1312, 1996; Carter and Merchant, Curr. Opin. Biotechnol. 8:449-454, 1997; Zuo et al., Protein Engineering 13:361-367, 2000; and Lu et al., J. Immunol. Methods 267:213-226, 2002.)

As used herein, the term “immunoglobulin” refers to a protein consisting of one or more polypeptides substantially encoded by immunoglobulin gene(s). One form of immunoglobulin constitutes the basic structural unit of native (i.e., natural) antibodies in vertebrates. This form is a tetramer and consists of two identical pairs of immunoglobulin chains, each pair having one light chain and one heavy chain. In each pair, the light and heavy chain variable regions (VL and VH) are together primarily responsible for binding to an antigen, and the constant regions are primarily responsible for the antibody effector functions. Five classes of immunoglobulin protein (IgG, IgA, IgM, IgD, and IgE) have been identified in higher vertebrates. IgG comprises the major class; it normally exists as the second most abundant protein found in plasma. In humans, IgG consists of four subclasses, designated IgG1, IgG2, IgG3, and IgG4. The heavy chain constant regions of the IgG class are identified with the Greek symbol γ. For example, immunoglobulins of the IgG1 subclass contain a γ1 heavy chain constant region. Each immunoglobulin heavy chain possesses a constant region that consists of constant region protein domains (CH1, hinge, CH2, and CH3; IgG3 also contains a CH4 domain) that are essentially invariant for a given subclass in a species. DNA sequences encoding human and non-human immunoglobulin chains are known in the art. (See, e.g., Ellison et al., DNA 1:11-18, 1981; Ellison et al., Nucleic Acids Res. 10:4071-4079, 1982; Kenten et al., Proc. Natl. Acad. Sci. USA 79:6661-6665, 1982; Seno et al., Nuc. Acids Res. 11:719-726, 1983; Riechmann et al., Nature 332:323-327, 1988; Amster et al., Nuc. Acids Res. 8:2055-2065, 1980; Rusconi and Kohler, Nature 314:330-334, 1985; Boss et al., Nuc. Acids Res. 12:3791-3806, 1984; Bothwell et al., Nature 298:380-382, 1982; van der Loo et al., Immunogenetics 42:333-341, 1995; Karlin et al., J. Mol. Evol. 22:195-208, 1985; Kindsvogel et al., DNA 1:335-343, 1982; Breiner et al., Gene 18:165-174, 1982; Kondo et al., Eur. J. Immunol. 23:245-249, 1993; and GenBank Accession No. J00228.) For a review of immunoglobulin structure and function, see Putnam, The Plasma Proteins, Vol V, Academic Press, Inc., 49-140, 1987; and Padlan, Mol. Immunol. 31:169-217, 1994. The term “immunoglobulin” is used herein for its common meaning, denoting an intact antibody, its component chains, or fragments of chains, depending on the context.

Full-length immunoglobulin “light chains” (about 25 Kd or 214 amino acids) are encoded by a variable region gene at the amino-terminus (encoding about 110 amino acids) and a by a kappa or lambda constant region gene at the carboxyl-terminus. Full-length immunoglobulin “heavy chains” (about 50 Kd or 446 amino acids) are encoded by a variable region gene (encoding about 116 amino acids) and a gamma, mu, alpha, delta, or epsilon constant region gene (encoding about 330 amino acids), the latter defining the antibody's isotype as IgG, IgM, IgA, IgD, or IgE, respectively. Within light and heavy chains, the variable and constant regions are joined by a “J” region of about 12 or more amino acids, with the heavy chain also including a “D” region of about 10 more amino acids. (See generally Fundamental Immunology (Paul, ed., Raven Press, N.Y., 2nd ed. 1989), Ch. 7).

An immunoglobulin light or heavy chain variable region (also referred to herein as a “light chain variable domain” (“VL domain”) or “heavy chain variable domain” (“VH domain”), respectively) consists of a “framework” region interrupted by three hypervariable regions, also called “complementarity determining regions” or “CDRs.” The framework regions serve to align the CDRs for specific binding to an epitope of an antigen. Thus, the term “hypervariable region” or “CDR” refers to the amino acid residues of an antibody that are primarily responsible for antigen binding. From amino-terminus to carboxyl-terminus, both VL and VH domains comprise the following framework (FR) and CDR regions: FR1, CDR1, FR2, CDR2, FR3, CDR3, FR4. The assignment of amino acids to each domain is in accordance with the definitions of Kabat, Sequences of Proteins of Immunological Interest (National Institutes of Health, Bethesda, Md., 1987 and 1991), or Chothia & Lesk, J. Mol. Biol. 196:901-917, 1987; Chothia et al., Nature 342:878-883, 1989. Kabat also provides a widely used numbering convention (Kabat numbering) in which corresponding residues between different heavy chains or between different light chains are assigned the same number. CDRs 1, 2, and 3 of a VL domain are also referred to herein, respectively, as CDR-L1, CDR-L2, and CDR-L3; CDRs 1, 2, and 3 of a VH domain are also referred to herein, respectively, as CDR-H1, CDR-H2, and CDR-H3.

Unless the context dictates otherwise, the term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

The term “chimeric antibody” refers to an antibody having variable domains derived from a first species and constant regions derived from a second species. Chimeric immunoglobulins or antibodies can be constructed, for example by genetic engineering, from immunoglobulin gene segments belonging to different species. The term “humanized antibody,” as defined infra, is not intended to encompass chimeric antibodies. Although humanized antibodies are chimeric in their construction (i.e., comprise regions from more than one species of protein), they include additional features (i.e., variable regions comprising donor CDR residues and acceptor framework residues) not found in chimeric immunoglobulins or antibodies, as defined herein.

The term “humanized VH domain” or “humanized VL domain” refers to an immunoglobulin VH or VL domain comprising some or all CDRs entirely or substantially from a non-human donor immunoglobulin (e.g., a mouse or rat) and variable region framework sequences entirely or substantially from human immunoglobulin sequences. The non-human immunoglobulin providing the CDRs is called the “donor” and the human immunoglobulin providing the framework is called the “acceptor.” In some instances, humanized antibodies may retain non-human residues within the human variable domain framework regions to enhance proper binding characteristics (e.g., mutations in the frameworks may be required to preserve binding affinity when an antibody is humanized).

A “humanized antibody” is an antibody comprising one or both of a humanized VH domain and a humanized VL domain. Immunoglobulin constant region(s) need not be present, but if they are, they are entirely or substantially from human immunoglobulin constant regions.

A CDR in a humanized antibody is “substantially from” a corresponding CDR in a non-human antibody when at least 60%, at least 85%, at least 90%, at least 95% or 100% of corresponding residues (as defined by Kabat) are identical between the respective CDRs. In particular variations of a humanized VH or VL domain in which CDRs are substantially from a non-human immunoglobulin, the CDRs of the humanized VH or VL domain have no more than six (e.g., no more than five, no more than four, no more than three, no more than two, or nor more than one) amino acid substitutions across all three CDRs relative to the corresponding non-human VH or VL CDRs. The variable region framework sequences of an antibody VH or VL domain or, if present, a sequence of an immunoglobulin constant region, are “substantially from” a human VH or VL framework sequence or human constant region, respectively, when at least 85%, at least 90%, at least 95%, or 100% of corresponding residues defined by Kabat are identical. Hence, all parts of a humanized antibody, except possibly the CDRs, are entirely or substantially from corresponding parts of natural human immunoglobulin sequences.

Specific binding of an antibody to its target antigen means an affinity of at least 10⁶, 10⁷, 10⁸, 10⁹, or 10¹⁰ M⁻¹. Specific binding is detectably higher in magnitude and distinguishable from non-specific binding occurring to at least one unrelated target. Specific binding can be the result of formation of bonds between particular functional groups or particular spatial fit (e.g., lock and key type) whereas nonspecific binding is usually the result of van der Waals forces. Specific binding does not, however, necessarily imply that a monoclonal antibody binds one and only one target.

With regard to proteins as described herein, reference to amino acid residues corresponding to those specified by SEQ ID NO includes post-translational modifications of such residues.

The term “anti-CD33 antibody” refers to an antibody that specifically binds to the human CD33 protein. In a preferred embodiment the anti-CD33 antibody comprises the CDRs of the light chain variable region of SEQ ID NO:1 and the CDRs of the heavy chain variable region of SEQ ID NO:2. In another preferred embodiment, the anti-CD33 antibody comprises the light chain variable region of SEQ ID NO:1 and the heavy chain variable region of SEQ ID NO:2. In other preferred embodiments the anti-CD33 antibody includes a human constant region and is an IgG1 antibody.

An antibody-drug conjugate (ADC) is an antibody conjugated to a cytotoxic drug typically via a linker. The linker may comprise a cleavable unit or may be non-cleavable. Cleavable units include, for example, disulfide containing linkers that are cleavable through disulfide exchange, acid-labile linkers that are cleavable at acidic pH, and linkers that are cleavable by hydrolases, esterases, peptidases, and glucoronidases (e.g., peptide linkers and glucoronide linkers). Non-cleavable linkers are believed to release drug via a proteolytic antibody degradation mechanism.

The term “diluent” as used herein refers to a solution suitable for altering or achieving an exemplary or appropriate concentration or concentrations as described herein.

The term “container” refers to something into which an object or liquid can be placed or contained, e.g., for storage (for example, a holder, receptacle, vessel, or the like).

The term “administration route” includes art-recognized administration routes for delivering a therapeutic protein such as, for example, parenterally, intravenously, intramuscularly, or subcutaneously. For administration of an ADC for the treatment of cancer, administration into the systemic circulation by intravenous or subcutaneous administration may be desired. For treatment of a cancer characterized by a solid tumor, administration can also be localized directly into the tumor, if so desired.

The term “treatment” refers to the administration of a therapeutic agent to a patient, who has a disease with the purpose to cure, heal, alleviate, delay, relieve, alter, remedy, ameliorate, improve or affect the disease.

The term “patient” includes human and other mammalian subjects that receive either prophylactic or therapeutic treatment.

The term “effective amount,” “effective dose,” or “effective dosage” refers to an amount that is sufficient to achieve or at least partially achieve the desired effect, e.g., sufficient to inhibit the occurrence or ameliorate one or more symptoms of a disease or disorder. An effective amount of a pharmaceutical composition is administered in an “effective regime.” The term “effective regime” refers to a combination of amount of the composition being administered and dosage frequency adequate to accomplish prophylactic or therapeutic treatment of the disease or disorder.

The term “dosage unit form” (or “unit dosage form”) as used herein refers to a physically discrete unit suitable as unitary dosages for a patient to be treated, each unit containing a predetermined quantity of active compound (an ADC in accordance with the present invention) calculated to produce the desired therapeutic effect in association with the required pharmaceutical carrier, diluent, or excipient. The specification for the dosage unit forms of the invention are dictated by and directly dependent on the unique characteristics of the active compound and the particular therapeutic effect to be achieved, and the limitations inherent in the art of compounding such an active compound for the treatment of patients.

Actual dosage levels of an ADC in a formulation of the present invention may be varied so as to obtain an amount of the ADC that is effective to achieve a desired therapeutic response for a particular patient, composition, and mode of administration, without being toxic to the patient. The selected dosage level will depend upon a variety of pharmacokinetic factors including the activity of the particular compositions of the present invention employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well-known in the medical arts.

The phrase “pharmaceutically acceptable salt,” as used herein, refers to pharmaceutically acceptable organic or inorganic salts of a compound. The compound can contain at least one amino group, and accordingly acid addition salts can be formed with the amino group. Exemplary salts include, but are not limited to, sulfate, trifluoroacetate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p toluenesulfonate, and pamoate (i.e., 1,1′ methylene bis-(2 hydroxy 3 naphthoate)) salts. A pharmaceutically acceptable salt may involve the inclusion of another molecule such as an acetate ion, a succinate ion or other counterion. The counterion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counterion.

A “cytotoxic effect” refers to the depletion, elimination and/or the killing of a target cell. A “cytotoxic agent” refers to an agent that has a cytotoxic effect on a cell.

A “cytostatic effect” refers to the inhibition of cell proliferation. A “cytostatic agent” refers to an agent that has a cytostatic effect on a cell, thereby inhibiting the growth and/or expansion of a specific subset of cells.

Two amino acid sequences have “100% amino acid sequence identity” if the amino acid residues of the two amino acid sequences are the same when aligned for maximal correspondence. Sequence comparisons can be performed using standard software programs such as those included in the LASERGENE bioinformatics computing suite, which is produced by DNASTAR (Madison, Wis.). Other methods for comparing two nucleotide or amino acid sequences by determining optimal alignment are well-known to those of skill in the art. (See, e.g., Peruski and Peruski, The Internet and the New Biology: Tools for Genomic and Molecular Research (ASM Press, Inc. 1997); Wu et al. (eds.), “Information Superhighway and Computer Databases of Nucleic Acids and Proteins,” in Methods in Gene Biotechnology 123-151 (CRC Press, Inc. 1997); Bishop (ed.), Guide to Human Genome Computing (2nd ed., Academic Press, Inc. 1998).) Two amino acid sequences are considered to have “substantial sequence identity” if the two sequences have at least 80%, at least 85%, at least 90%, or at least 95% sequence identity relative to each other.

Percentage sequence identities are determined with antibody sequences maximally aligned by the Kabat numbering convention. After alignment, if a subject antibody region (e.g., the entire variable domain of a heavy or light chain) is being compared with the same region of a reference antibody, the percentage sequence identity between the subject and reference antibody regions is the number of positions occupied by the same amino acid in both the subject and reference antibody region divided by the total number of aligned positions of the two regions, with gaps not counted, multiplied by 100 to convert to percentage.

The term “pharmaceutical formulation” refers to a preparation which is in such form as to permit the biological activity of the active ingredient to be effective (when administered to a subject), and which contains no additional components which are unacceptably toxic to a subject to which the formulation would be administered. Such formulations are sterile.

Compositions or methods “comprising” one or more recited elements may include other elements not specifically recited.

Reference to a numerical range herein (e.g., “X to Y” or “from X to Y”) includes the endpoints defining the range and all values falling within the range.

As used herein, the term “about” denotes an approximate range of plus or minus 10% from a specified value. For instance, the language “about 20%” encompasses a range of 18-22%. As used herein, about also includes the exact amount. Hence “about 20%” means “about 20%” and also “20%.”

### DETAILED DESCRIPTION

This invention demonstrates optimal dosing of SGN-CD33 A, a CD33-antibody drug conjugate (CD33-ADC), i.e., h2H12 antibody conjugated to a PBD, with the hypomethylating agents 5-azacytidine or 5-aza-2-deoxycytidine.

I. CD33 Antibody Drug Conjugates

A. Anti-CD33 Antibodies

The anti-CD33 antibody disclosed herein is the humanized 2H12 antibody (h2H12). The murine 2H12 antibody was raised in mice, using the human CD33 protein as an immunogen. After making hybridomas from the spleens of the immunized mice, followed by screening for CD33 binding activity, the murine 2H12 antibody was selected for humanization. The h2H12 antibody was derived from the murine 2H12 antibody. The humanization procedure is disclosed in PCT publication WO 2013/173,496; which is herein incorporated by reference for all purposes. The variable region sequences of the h2H12 light and heavy chains are provided as SEQ ID NO:1 and SEQ ID NO:2, respectively.

The h2H12 antibody comprises human constant regions. Sequences of human constant regions are provided in the sequence listing. The heavy chain constant region of h2H12 includes a substitution mutation, S239C, to facilitate conjugation of a drug-linker to the antibody. The sequence of a human constant region comprising the S239C mutation is provided at SEQ ID NOs:6 and 7. The h2H12 antibody comprising the S239C mutation is also referred to as h2H12EC.

B. Drug Linkers

Exemplary CD33 antibody-drug conjugates include PBD based antibody-drug conjugates; i.e., antibody-drug conjugates wherein the drug component is a PBD drug.

PBDs are of the general structure:

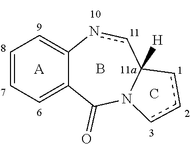

They differ in the number, type and position of substituents, in both their aromatic A rings and pyrrolo C rings, and in the degree of saturation of the C ring. In the B-ring there is either an imine (N═C), a carbinolamine (NH—CH(OH)), or a carbinolamine methyl ether (NH—CH(OMe)) at the N10-C11 position, which is the electrophilic centre responsible for alkylating DNA. All of the known natural products have an (S)-configuration at the chiral C11a position which provides them with a right-handed twist when viewed from the C ring towards the A ring. This gives them the appropriate three-dimensional shape for isohelicity with the minor groove of B-form DNA, leading to a snug fit at the binding site (Kohn, In Antibiotics III. Springer-Verlag, New York, pp. 3-11 (1975); Hurley and Needham-VanDevanter, Acc. Chem. Res., 19, 230-237 (1986)). The ability of PBDs to form an adduct in the minor groove enables them to interfere with DNA processing, hence their use as antitumour agents.

The biological activity of these molecules can be potentiated by joining two PBD units together through their C8/C′-hydroxyl functionalities via a flexible alkylene linker (Bose, D. S., et al., J. Am. Chem. Soc., 114, 4939-4941 (1992); Thurston, D. E., et al., J. Org. Chem., 61, 8141-8147 (1996)). The PBD dimers are thought to form sequence-selective DNA lesions such as the palindromic 5′-Pu-GATC-Py-3′ interstrand cross-link (Smellie, M., et al., Biochemistry, 42, 8232-8239 (2003); Martin, C., et al., Biochemistry, 44, 4135-4147) which is thought to be mainly responsible for their biological activity.

In some embodiments, PBD based antibody-drug conjugates comprise a PBD dimer linked to an anti-CD33 antibody. The monomers that form the PBD dimer can be the same or different, i.e., symmetrical or unsymmetrical. The PBD dimer can be linked to the anti-CD33 antibody at any position suitable for conjugation to a linker. For example, in some embodiments, the PBD dimer will have a substituent at the C2 position that provides an anchor for linking the compound to the anti-CD33 antibody. In alternative embodiments, the N10 position of the PBD dimer will provide the anchor for linking the compound to the anti-CD33 antibody.

Typically the PBD based antibody-drug conjugate comprises a linker between the PBD drug and the anti-CD33 antibody. The linker may comprise a cleavable unit (e.g., an amino acid or a contiguous sequence of amino acids that is a target substrate for an enzyme) or a non-cleavable linker (e.g., linker released by degradation of the antibody). The linker may further comprise a maleimide group for linkage to the antibody, e.g., maleimidocaproyl. The linker may, in some embodiments, further comprise a self-immolative group, such as, for example, a p-aminobenzyl alcohol (PAB) unit.

An exemplary PBD for use as a conjugate is described in International Application No. WO 2011/130613 and is as follows wherein the wavy line indicates the site of attachment to the linker:

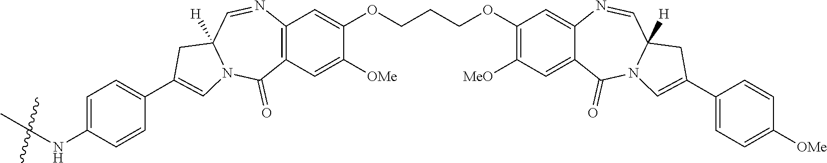 [SMILES: *Nc1ccc(C2=CN3C(=O)c4cc(OC)c(OCCCOc5cc6c(cc5OC)C(=O)N5C=C(c7ccc(OC)cc7)C[C@H]5C=N6)cc4N=C[C@@H]3C2)cc1]

or a pharmaceutically acceptable salt thereof. An exemplary linker is as follows wherein the wavy line indicates the site of attachment to the drug and the antibody is linked via the maleimide group.

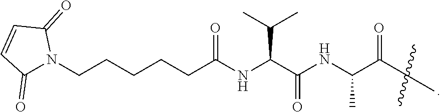 [SMILES: *C(=O)[C@H](C)NC(=O)[C@@H](NC(=O)CCCCCN1C(=O)C=CC1=O)C(C)C]

Exemplary PBDs based antibody-drug conjugates include antibody-drug conjugates as shown below wherein Ab is an antibody as described herein:

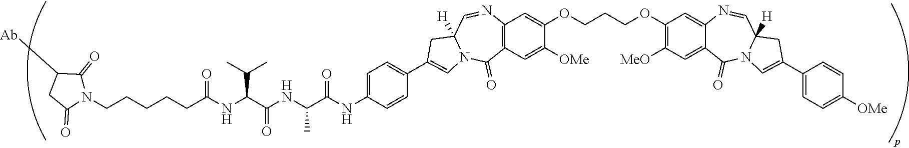

or a pharmaceutically acceptable salt thereof. The drug loading is represented by p, the number of drug-linker molecules per antibody. Depending on the context, p can represent the average number of drug-linker molecules per antibody, also referred to the average drug loading. The variable p ranges from 1 to 20 and is preferably from 1 to 8. In some preferred embodiments, when p represents the average drug loading, p ranges from about 2 to about 5. In some embodiments, p is about 2, about 3, about 4, or about 5. In some aspects, the antibody is conjugated to the drug linker via a sulfur atom of a cysteine residue that is engineered into the antibody. In some aspects, the cysteine residue is engineered into the antibody at position 239 (IgG1) as determined by the EU index (Kabat, Sequences of Proteins of Immunological Interest (National Institutes of Health, Bethesda, Md., 1987 and 1991).

C. CD33-ADCs

As used herein a “CD33-ADC” refers to an ADC that comprises an h2H12 antibody conjugated to a PBD molecule. The antibody portion comprises the variable light chain region of SEQ ID NO:1 and the variable heavy chain region of SEQ ID NO:2. The constant region is a human IgG1 constant region. The heavy chain constant region has a substitution mutation at amino acid 239 using Kabat numbering, i.e., S239C. The cysteine residue at position 239 is the point of attachment for the PBD molecule. The structure of the antibody, the linker and the PBD molecule is shown above. Methods to make the CD33-ADC are disclosed in PCT publication WO 2013/173,496 and PCT publication WO 2011/130613, both of which are incorporated by reference for all purposes.

II. Hypomethylating Agents

Hypomethylating agents inhibit DNA methylation by inhibiting the activity of the DNA methyltransferases. Exemplary hypomethylating agents are 5-azacytidine (VIDAZA®) and 5-aza-2-deoxycytidine or decitabine (DACOGEN®). Both compounds are cytidine analogs, approved drugs, and are commercially available. 5-azacytidine is incorporated in to both DNA and RNA. See, e.g., Raj and Mufti Thera. and Clin. Risk Manag. 2:377 (2006). Once in DNA, it binds irreversibly to DNA methyltransferases, thereby blocking DNA methylation. Decitabine is incorporated only into DNA, but acts through a similar mechanism. See, e.g., ibid.

III. Treatment of Acute Myeloid Leukemia (AML)

CD33-ADCs in combination with hypomethylating agents can be used to treat acute myeloid leukemia (AML), preferably AML that has detectable levels of CD33 measured at either the protein (e.g., by immunoassay using one of the exemplified antibodies) or mRNA level. Some such AML cells show elevated levels of CD33 relative to noncancerous tissue of the same type, preferably from the same patient. An exemplary level of CD33 on AML samples amenable to treatment is 5000-150000 CD33 molecules per cell, although higher or lower levels can be treated. Optionally, a level of CD33 in a cancer is measured before performing treatment.

The combination of CD33-ADC with a hypomethylating agent treatment can be applied to patients who are treatment naïve, who are refractory to conventional treatments (e.g., chemotherapy or MYLOTARG® (gemtuzumab ozogamicin), or who have relapsed following a response to such treatments. Some cancer cells develop resistance to a therapeutic agent after increasing expression of a protein increases efflux of the therapeutic agent out of the cancer cell. Such proteins include P-glycoprotein, multidrug resistance-associated protein, lung resistance-related protein, and breast cancer resistance protein. Detection of drug resistance in cancer cells can be performed by those of skill. Antibodies or assays that detect efflux proteins are commercially available from, e.g., Promega, Millipore, Abcam, and Sigma-Aldrich. In one embodiment, a CD33-ADC in combination with a hypomethylating agent is used to treat a subject with a multi-drug resistant, CD33-positive AML.

In some embodiments the combination of a CD33-ADC with a hypomethylating agent is used to treat elderly patients, e.g., patients 60 years old or older who have CD33 positive AML. In other embodiments the combination of a CD33-ADC with a hypomethylating agent is used to treat elderly patients, e.g., patients 75 years old or older who have CD33 positive AML. In further embodiments the combination of a CD33-ADC with a hypomethylating agent is used to treat elderly patients, e.g., patients 75-100 years old who have CD33 positive AML, patients 75-90 years old who have CD33 positive AML, or patients 75-85 years old who have CD33 positive AML. Other frail or unfit patients can be treated using the combination of a CD33-ADC and a hypomethylating agent, for example, patients that decline or who are not candidates for standard induction/consolidation treatment. Additionally, elderly patients with poor risk disease characteristics can also be treated using the combination, given the lack of benefit observed with intensive chemotherapy. Poor disease risk characteristics are known and described at, e.g., Hou et al., Leukemia 28:50-58 (2014).

In another embodiment, the combination of a CD33-ADC with a hypomethylating agent is used to treat fit, non-elderly patients, i.e., patients younger than 75 years old who have difficult to treat forms of AML. Difficult to treat forms of AML include, e.g., relapsed or refractory AML or secondary AML. Secondary AML is associated with exposure to a leukemogenic agent, e.g., previous chemotherapy or radiotherapy, some immunosuppressive drugs or environmental leukemogenic agents).

IV. Dosage and Administration

Pharmaceutical compositions for parenteral administration are preferably sterile and substantially isotonic and manufactured under GMP conditions. Pharmaceutical compositions can be provided in unit dosage form (i.e., the dosage for a single administration). Pharmaceutical compositions can be formulated using one or more physiologically acceptable carriers, diluents, excipients or auxiliaries. The formulation depends on the route of administration chosen. For injection, antibodies can be formulated in aqueous solutions, preferably in physiologically compatible buffers such as Hank's solution, Ringer's solution, or physiological saline or acetate buffer (to reduce discomfort at the site of injection). The solution can contain formulatory agents such as suspending, stabilizing and/or dispersing agents. Alternatively antibodies can be in lyophilized form for constitution with a suitable vehicle, e.g., sterile pyrogen-free water, before use. Formulations for the CD33-ADC comprising h2H12 antibody and a PBD molecule are disclosed e.g., at PCT/US2014/024466.

The CD33-ADC is administered intravenously, as is decitabine. 5-Azacytidine can be administered intravenously or subcutaneously.

The CD33-ADC can be combined with a hypomethylating agent concurrently or sequentially for treatment of a CD33-expressing cancer or disorder, e.g., AML or a myelodysplastic syndrome, at the discretion of the treating physician.

In one embodiment, the combination of the CD33-ADC with 5-azacytidine is dosed on a twenty-eight day schedule. 5-azacytidine is administered on days 1-7 or on days 1-5, followed by two days off, and two more days of 5-azacytidine. In one embodiment, 10 μg/kg CD33-ADC is administered on the final day of 5-azacytidine treatment. After day 28, the cycle is repeated, with the total number of cycles determined by the physician.

In one embodiment, combination of the CD33-ADC with decitabine is dosed on a twenty-eight day schedule. Decitabine is administered on days 1-5. In one embodiment, 10 μg/kg CD33-ADC is administered on the final day of decitabine treatment, e.g., day 5. After day 28, the cycle is repeated, with the total number of cycles determined by the physician.

The CD33-ADC can be administered in combination with 5-azacytidine or decitabine in the following dose ranges: about 10 μg/kg CD33-ADC in combination with 5-azacytidine or decitabine. In another embodiment, the CD33-ADC is administered at 10 μg/kg in combination with 5-azacytidine or decitabine.

5-Azacytidine can be administered in the following dose ranges in combination with the CD33-ADC: 10-200 mg/m², 25-150 mg/m², or 50-100 mg/m². In some embodiments, 5-azacytidine is administered at about 75 mg/m² in combination with the CD33-ADC. In another embodiment, 5-azacytidine is administered at 75 mg/m² in combination with the CD33-ADC. Decitabine can be administered in the following dose ranges in combination with the CD33-ADC: 5-50 mg/m², 10-30 mg/m², or 15-25 mg/m². In some embodiments, decitabine is administered at about 20 mg/m² in combination with the CD33-ADC. In another embodiment, decitabine is administered at 20 mg/m² in combination with the CD33-ADC.

In one embodiment, the combination of the CD33-ADC with 5-azacytidine is dosed on a twenty-eight day schedule. 5-azacytidine is administered at 75 mg/m² on days 1-7 or on days 1-5, followed by two days off, and two more days of 5-azacytidine at 75 mg/m². The CD33-ADC is administered at about 10 μg/kg on the final day of 5-azacytidine treatment. After day 28, the cycle is repeated, with the total number of cycles determined by the physician.

In one embodiment, the combination of the CD33-ADC with 5-azacytidine is dosed on a twenty-eight day schedule. 5-azacytidine is administered at 75 mg/m² on days 1-7 or on days 1-5, followed by two days off, and two more days of 5-azacytidine at 75 mg/m². The CD33-ADC is administered at 10 μg/kg on the final day of 5-azacytidine treatment. After day 28, the cycle is repeated, with the total number of cycles determined by the physician.

In one embodiment, combination of the CD33-ADC with decitabine is dosed on a twenty-eight day schedule. Decitabine is administered at 20 mg/m² on days 1-5. The CD33-ADC is administered at about 10 μg/kg on the final day of decitabine treatment, e.g., day 5. After day twenty-eight, the cycle is repeated, with the total number of cycles determined by the physician.

In one embodiment, combination of the CD33-ADC with decitabine is dosed on a twenty-eight day schedule. Decitabine is administered at 20 mg/m² on days 1-5. The CD33-ADC is administered at 10 μg/kg on the final day of decitabine treatment, e.g., day 5. After day twenty-eight, the cycle is repeated, with the total number of cycles determined by the physician.

In one embodiment, the combination of the CD33-ADC with 5-azacytidine is dosed on a twenty-eight day schedule for a patient with CD33 positive AML who is 75 years old or older. 5-azacytidine is administered at 75 mg/m² on days 1-7 or on days 1-5, followed by two days off, and two more days of 5-azacytidine at 75 mg/m². The CD33-ADC is administered at about 10 μg/kg on the final day of 5-azacytidine treatment. After day 28, the cycle is repeated, with the total number of cycles determined by the physician.

In one embodiment, the combination of the CD33-ADC with 5-azacytidine is dosed on a twenty-eight day schedule for a patient with CD33 positive AML who is 75 years old or older. 5-azacytidine is administered at 75 mg/m² on days 1-7 or on days 1-5, followed by two days off, and two more days of 5-azacytidine at 75 mg/m². The CD33-ADC is administered at 10 μg/kg on the final day of 5-azacytidine treatment. After day 28, the cycle is repeated, with the total number of cycles determined by the physician.

In one embodiment, combination of the CD33-ADC with decitabine is dosed on a twenty-eight day schedule for a patient with CD33 positive AML who is 75 years old or older. Decitabine is administered at 20 mg/m² on days 1-5. The CD33-ADC is administered at about 10 μg/kg on the final day of decitabine treatment, e.g., day 5. After day twenty-eight, the cycle is repeated, with the total number of cycles determined by the physician.

In one embodiment, combination of the CD33-ADC with decitabine is dosed on a twenty-eight day schedule for a patient with CD33 positive AML who is 75 years old or older. Decitabine is administered at 20 mg/m² on days 1-5. The CD33-ADC is administered at 10 μg/kg on the final day of decitabine treatment, e.g., day 5. After day twenty-eight, the cycle is repeated, with the total number of cycles determined by the physician.

In one embodiment, combination of the CD33-ADC with decitabine is dosed on a twenty-eight day schedule for a patient with CD33 positive secondary AML who is younger than 75 years old. Decitabine is administered at 20 mg/m² on days 1-5. The CD33-ADC is administered at between 20-40 μg/kg on the final day of decitabine treatment, e.g., day 5. This dose cycle is repeated for up to 4 cycles. At cycle five, the CD33-ADC is administered at 10 μg/kg on the final day of decitabine treatment, e.g., day 5. Cycle 5 dosing is repeated, with the total number of cycles determined by the physician.

In one embodiment, combination of the CD33-ADC with decitabine is dosed on a twenty-eight day schedule for a patient with CD33 positive secondary AML who is younger than 75 years old. Decitabine is administered at 20 mg/m² on days 1-5. The CD33-ADC is administered at 20 μg/kg on the final day of decitabine treatment, e.g., day 5. This dose cycle is repeated for up to 4 cycles. At cycle five, the CD33-ADC is administered at 10 μg/kg on the final day of decitabine treatment, e.g., day 5. Cycle 5 dosing is repeated, with the total number of cycles determined by the physician.

In one embodiment, combination of the CD33-ADC with decitabine is dosed on a twenty-eight day schedule for a patient with CD33 positive secondary AML who is younger than 75 years old. Decitabine is administered at 20 mg/m² on days 1-5. The CD33-ADC is administered at 30 μg/kg on the final day of decitabine treatment, e.g., day 5. This dose cycle is repeated for up to 4 cycles. At cycle five, the CD33-ADC is administered at 10 μg/kg on the final day of decitabine treatment, e.g., day 5. Cycle 5 dosing is repeated, with the total number of cycles determined by the physician.

In one embodiment, combination of the CD33-ADC with decitabine is dosed on a twenty-eight day schedule for a patient with CD33 positive secondary AML who is younger than 75 years old. Decitabine is administered at 20 mg/m² on days 1-5. The CD33-ADC is administered at 40 μg/kg on the final day of decitabine treatment, e.g., day 5. This dose cycle is repeated for up to 4 cycles. At cycle five, the CD33-ADC is administered at 10 μg/kg on the final day of decitabine treatment, e.g., day 5. Cycle 5 dosing is repeated, with the total number of cycles determined by the physician.

In one embodiment, combination of the CD33-ADC with decitabine is dosed on a twenty-eight day schedule for a patient with CD33 positive relapsed/refractory AML who is younger than 75 years old. Decitabine is administered at 20 mg/m² on days 1-5. The CD33-ADC is administered at between 20-40 μg/kg on the final day of decitabine treatment, e.g., day 5. This dose cycle is repeated for up to 4 cycles. At cycle five, the CD33-ADC is administered at 10 μg/kg on the final day of decitabine treatment, e.g., day 5. Cycle 5 dosing is repeated, with the total number of cycles determined by the physician.

In one embodiment, combination of the CD33-ADC with decitabine is dosed on a twenty-eight day schedule for a patient with CD33 positive relapsed/refractory AML who is younger than 75 years old. Decitabine is administered at 20 mg/m² on days 1-5. The CD33-ADC is administered at 20 μg/kg on the final day of decitabine treatment, e.g., day 5. This dose cycle is repeated for up to 4 cycles. At cycle five, the CD33-ADC is administered at 10 μg/kg on the final day of decitabine treatment, e.g., day 5. Cycle 5 dosing is repeated, with the total number of cycles determined by the physician.

In one embodiment, combination of the CD33-ADC with decitabine is dosed on a twenty-eight day schedule for a patient with CD33 positive relapsed/refractory AML who is younger than 75 years old. Decitabine is administered at 20 mg/m² on days 1-5. The CD33-ADC is administered at 30 μg/kg on the final day of decitabine treatment, e.g., day 5. This dose cycle is repeated for up to 4 cycles. At cycle five, the CD33-ADC is administered at 10 μg/kg on the final day of decitabine treatment, e.g., day 5. Cycle 5 dosing is repeated, with the total number of cycles determined by the physician.

In one embodiment, combination of the CD33-ADC with decitabine is dosed on a twenty-eight day schedule for a patient with CD33 positive relapsed/refractory AML who is younger than 75 years old. Decitabine is administered at 20 mg/m² on days 1-5. The CD33-ADC is administered at 40 μg/kg on the final day of decitabine treatment, e.g., day 5. This dose cycle is repeated for up to 4 cycles. At cycle five, the CD33-ADC is administered at 10 μg/kg on the final day of decitabine treatment, e.g., day 5. Cycle 5 dosing is repeated, with the total number of cycles determined by the physician.

### EXAMPLES

The following examples are offered to illustrate, but not to limit the claimed invention.

### Example 1: CD33-ADC in Combination with Hypomethylating Agents is Well Tolerated and Elicits a High Remission Rate in Older Patients with AML

### Methods

A combination cohort in a phase 1 study (NCT01902329) was designed to evaluate the safety, tolerability, pharmacokinetics (PK), and anti-leukemic activity of 33 A in combination with an HMA. Eligible patients (ECOG 0-1) must have previously untreated CD33-positive AML, and have declined intensive therapy. A single dose level of 33 A, 10 mcg/kg, was administered outpatient IV every 4 weeks on the last day of HMA (azacitidine or decitabine [5 day regimen], standard dosing). Patients with clinical benefit may continue treatment until relapse or unacceptable toxicity. Investigator assessment of response is per IWG criteria; CRi requires either platelet count of ≥100,000/μL or neutrophils of ≥1,000/μL (Cheson 2003).

### Results

To date, 24 patients (63% male) with a median age of 77 years (range, 66-83) have been treated with the combination therapy. 42% of patients had adverse cytogenetics (MRC), 23 patients were treatment naïve and 1 patient had received prior low intensity therapy for myelodysplastic syndrome (MDS). At baseline, patients had a median of 60% BM blasts (range, 2%-90%) and a median of WBC of 2.2 (range, 1-132). At the time of this interim analysis, patients were on treatment for a median of 13.5+ weeks with 17 patients continuing treatment; no DLTs have been reported. Grade 3 or higher adverse events (AE) reported in >20% of patients were fatigue (54%), febrile neutropenia (46%), anemia (25%), neutropenia (25%), and thrombocytopenia (21%). Other treatment-emergent AEs regardless of relationship to study treatment reported in >20% of patients were nausea (29%), decreased appetite (25%), and constipation (21%). Thirty- and 60-day mortality rates are 0% and 4% respectively with no treatment-related deaths reported. Fifteen of the 23 efficacy evaluable patients (65%) achieved CR (5) or CRi (10). Remissions were generally obtained after 2 cycles of treatment and were observed in many patients with adverse risk including underlying myelodysplasia (6/8, 75%) and adverse cytogenetics (8/9, 89%). Median OS has not been reached with 20 patients alive at the time of this data cut.

The combination of 33 A with HMA appears to be well-tolerated, active, and has no identified off-target toxicities. Activity with the combination compares favorably with historical experience with HMAs alone in this patient population.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method of treating CD33 expressing acute myeloid leukemia (AML) in a subject in need of such treatment, the method comprising the step of administering a hypomethylating agent and 10 μg/kg of a CD33 antibody drug conjugate (ADC), wherein the CD33-ADC comprises a humanized 2H12 antibody and a PBD cytotoxic agent, and wherein the hypomethylating agent is selected from the group consisting of 5-azacytidine and 5-aza-2-deoxycytidine.

2. The method of claim 1, wherein the PBD cytotoxic agent has the formula

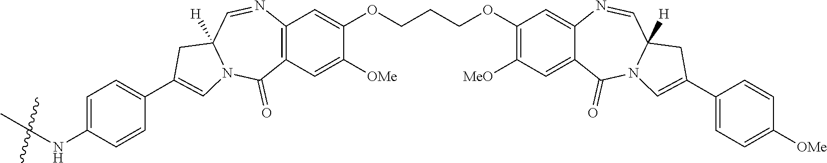 [SMILES: *Nc1ccc(C2=CN3C(=O)c4cc(OC)c(OCCCOc5cc6c(cc5OC)C(=O)N5C=C(c7ccc(OC)cc7)C[C@H]5C=N6)cc4N=C[C@@H]3C2)cc1]

3. The method of claim 1, wherein the hypomethylating agent is 5-azacytidine.

4. The method of claim 3, wherein the 5-azacytidine is administered at a concentration between 50-100 mg/m².

5. The method of claim 3, wherein the 5-azacytidine is administered at a concentration of about 75 mg/m².

6. The method of claim 4, wherein the 5-azacytidine is administered at a concentration of 75 mg/m².

7. The method of claim 1, wherein the hypomethylating agent is 5-aza-2-deoxycytidine.

8. The method of claim 7, wherein the 5-aza-2-deoxycytidine is administered at a concentration between 15-25 mg/m².

9. The method of claim 7, wherein the 5-aza-2-deoxycytidine is administered at a concentration of about 20 mg/m².

10. The method of claim 7, wherein the 5-aza-2-deoxycytidine is administered at a concentration of 20 mg/m².

11. The method of claim 1, wherein the subject is 75 years or older.

12. A method of treating CD33-expressing secondary acute myeloid leukemia (AML) in a subject in need of such treatment, the method comprising the step of administering a hypomethylating agent and between 20-40 μg/kg of a CD33 antibody drug conjugate (ADC), wherein the CD33-ADC comprises a humanized 2H12 antibody and a PBD cytotoxic agent, and wherein the hypomethylating agent is 5-aza-2-deoxycytidine.

13. The method of claim 12, wherein the PBD cytotoxic agent has the formula

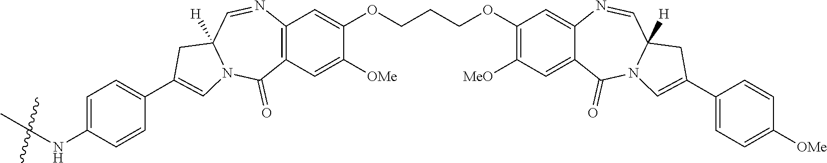 [SMILES: *Nc1ccc(C2=CN3C(=O)c4cc(OC)c(OCCCOc5cc6c(cc5OC)C(=O)N5C=C(c7ccc(OC)cc7)C[C@H]5C=N6)cc4N=C[C@@H]3C2)cc1]

14. The method of claim 12, wherein the 5-aza-2-deoxycytidine is administered at a concentration of about 20 mg/m².

15. The method of claim 12, wherein the 5-aza-2-deoxycytidine is administered at a concentration of 20 mg/m².

16. The method of claim 12, wherein the 5-aza-2-deoxycytidine is administered at a concentration of about 20 mg/m².

17. The method of claim 12, wherein the CD33-ADC is administered at 20 μg/kg.

18. The method of claim 12, wherein the CD33-ADC is administered at 30 μg/kg.

19. The method of claim 12, wherein the CD33-ADC is administered at 40 μg/kg.

20. The method of claim 12, wherein the subject is younger than 75 years old.

21. A method of treating CD33-expressing relapsed or refractory acute myeloid leukemia (AML) in a subject in need of such treatment, the method comprising the step of administering a hypomethylating agent and between 20-40 μg/kg of a CD33 antibody drug conjugate (ADC), wherein the CD33-ADC comprises a humanized 2H12 antibody and a PBD cytotoxic agent, and wherein the hypomethylating agent is 5-aza-2-deoxycytidine.

22. The method of claim 21, wherein the PBD cytotoxic agent has the formula

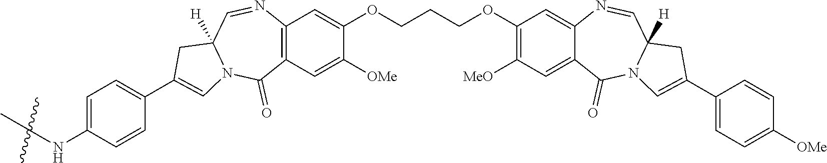 [SMILES: *Nc1ccc(C2=CN3C(=O)c4cc(OC)c(OCCCOc5cc6c(cc5OC)C(=O)N5C=C(c7ccc(OC)cc7)C[C@H]5C=N6)cc4N=C[C@@H]3C2)cc1]

23. The method of claim 21, wherein the 5-aza-2-deoxycytidine is administered at a concentration of about 20 mg/m².

24. The method of claim 21, wherein the 5-aza-2-deoxycytidine is administered at a concentration of 20 mg/m².

25. The method of claim 21, wherein the 5-aza-2-deoxycytidine is administered at a concentration of about 20 mg/m².

26. The method of claim 21, wherein the CD33-ADC is administered at 20 μg/kg.

27. The method of claim 21, wherein the CD33-ADC is administered at 30 μg/kg.

28. The method of claim 21, wherein the CD33-ADC is administered at 40 μg/kg.

29. The method of claim 21, wherein the subject is younger than 75 years old.


SEQUENCE LISTING (7 sequences)

SEQ ID NO: 1 - protein, 107 aa, Artificial Sequence
  2H12 LG  Light chain variable region
DIQMTQSPSSLSASVGDRVTINCKASQDINSYLSWFQQKPGKAPKTLIYRANRLVDGVPSRFSGSGSGQDYTLTISSLQPEDFATYYCLQYDEFPLTFGGGTKVEIK

SEQ ID NO: 2 - protein, 117 aa, Artificial Sequence
  2H12 HI  Heavy chain variable region
QVQLVQSGAEVKKPGASVKVSCKASGYTFTNYDINWVRQAPGQGLEWIGWIYPGDGSTKYNEKFKAKATLTADTSTSTAYMELRSLRSDDTAVYYCASGYEDAMDYWGQGTTVTVSS

SEQ ID NO: 3 - protein, 106 aa, Homo sapiens
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 330 aa, Homo sapiens [sequence omitted]

SEQ ID NO: 5 - protein, 329 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 6 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 329 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG